(19) United States
(12) Patent Application Publication
(10) Pub. No.: US 2010/0189705 A1
(43) Pub. Date: Jul. 29, 2010

(54) BIOLOGICAL DECONTAMINATION SYSTEM

(76) Inventors: Stephen B. Squires, Carson City, NV (US); David C. Doderer, Carson City, NV (US); Anne C. Regina, Exton, PA (US); Bryan E. Rambo, Glenmoore, PA (US); Ashley A.G. Price, Exton, PA (US); John E. Hughes, Exton, PA (US)

Correspondence Address: HECKER LAW GROUP, PLC, SUITE 2300, 1925 CENTURY PARK EAST, LOS ANGELES, CA, 90067, US

(21) Appl. No.: 11/916,250

(22) PCT Filed: May 31, 2006
(86) PCT No.: PCT/US06/20761

§ 371 Date: Nov. 30, 2007

(51) Int. Cl. A61K 38/44; A61K 38/46; A61K 38/48

(52) U.S. Cl. 424/94.4; 424/94.6; 424/94.63

(57) ABSTRACT

A composition for decontaminating biological pathogens and a system and method for mixing and applying the composition to contaminated sites are presented. The composition of the invention is suitable for the decontamination of biological warfare agents, including bacillus anthracis, and is suited for wide area or large surface decontamination. In one or more embodiments, the composition includes a blend of biocides, surfactants, a basing component. In one or more embodiments, the composition additionally includes a protein and/or an enzyme. In one or more embodiments, the composition includes a foam forming material for effective application over large surfaces. In one or more embodiments, the composition is mixed on-site from a plurality of pre-mixed components to form a foam composition for application to a chemically or biologically contaminated site.

### FIELD OF THE INVENTION

This invention relates to the field of biological and chemical decontamination systems, and more specifically to a composition and method for decontaminating chemical agents and biological pathogens.

A portion of the disclosure of this patent document contains material that is subject to copyright protection. The copyright owner has no objection to the facsimile reproduction by anyone of the patent document or the patent disclosure, as it appears in the Patent and Trademark Office file or records, but otherwise reserves all copyrights associated with this document.

### BACKGROUND

There exists a risk of purposeful or accidental release of chemical and/or biological warfare agents that can cause serious harm to persons and the environment. To counteract such a release, there exists a need for non-hazardous compositions that can be applied to a contaminated area to decompose biological and/or chemical warfare agents.

One proposed chemical/biological decontamination composition is disclosed in Conerly et al., U.S. patent application Ser. No. 10/182821, published as U.S. Patent Publication No. 20030109017. Conerly et al. discloses a composition comprising a blend of three biocides (triclosan, benzalkonium chloride (BAC), and tetrakishydroxymethyl phosphonium sulfate (THPS)), an enzyme, a protein, and a buffered foam forming material. The preferred biocide blend disclosed by Conerly et al. contains about 0.5% by weight triclosan, about 0.5% by weight of BAC, and about 1.5% by weight of THPS. Conerly et al. claims that the disclosed composition is effective as a biological and chemical contaminant for a number of biological and chemical pathogens, including Sarin, VX, mustard gas, Anthrax (Bacillus anthracis or “B. anthracis”), the plague, cholera, tularemaia, E-coli, and Shigella. The Conerly et al. composition, however, does not produce a satisfactory level of decontamination for certain decontamination situations. Further, its foaming characteristics and corrosivity makes it unsuitable for use in some environments.

A problem with chemical/biological decontamination compositions is the difficulty of forming of a foam suitable for spraying or application to a contaminated site. Some ingredients, particularly the biocide benzalkonium chloride (BAC), inhibit foam formation. Being able to deliver a decontaminant in foam form is essential to ensure a sufficient level and degree of coverage of a site and to prevent runoff of contaminants. It would be desirable to have a decontamination composition that has BAC and that exhibits a sufficient degree of foam formation.

There remains a need for a biological and chemical decontaminant that is non-toxic, non-corrosive, easy and safe to apply, and that is effective against Anthrax and other biological and chemical pathogens.

### SUMMARY OF THE INVENTION

A composition for decontaminating biological and chemical pathogens and a system and method for preparing and delivering the composition onto a contaminated site are presented. The composition of the invention is particularly suitable for the decontamination of biological warfare agents, including bacillus anthracis, and is especially suited for wide area or large surface decontamination. In one or more embodiments, the composition includes a soluble combination of biocides, surfactants, a basing component, and may additionally include a protein and/or an enzyme that provide chemical decontamination capabilities. In one or more embodiments, the composition includes a foam forming material for effective application over large surfaces. In one or more embodiments, the composition is non-toxic and non-corrosive. In one or more embodiments, the composition is mixed on-site from a plurality pre-mixed components to form a foam composition for application to a chemically or biologically contaminated site. In one or more embodiments, the composition includes benzalkonium chloride, a basing agent, an anionic surfactant (present at about 0.8 wt % or more based on the total weight of the composition), and water. In one or more embodiments, the composition has a pH of about 6 to about 9.

The present invention also provides a method for treating a chemically or biologically contaminated site. The method includes the step of applying a foam composition having benzalkonium chloride, a basing agent, an anionic surfactant present at about 0.8 wt % or more based on the total weight of the composition, and water to the site. The foam composition has a pH of about 6 to about 9.

### DETAILED DESCRIPTION

A composition, system and method useful for decontaminating biological and chemical pathogens are presented. As used herein, the term “biological pathogen” includes any microorganism or toxin derived from a microorganism that causes disease in man, plants, or animals, including biological warfare agents.

The composition of the present invention is useful in a variety of applications where biological contamination may be of concern. The present invention is particularly suitable for use against biological warfare agents and naturally occurring biological pathogens.

In one or more embodiments, the composition of the present invention comprises generally a soluble combination of a biocide component, a basing component and a protein and/or an enzyme. In one or more embodiments, the pH range of the composition is adjusted to be between 7 and 9.

In one or more embodiments, the biocide component comprises a blend of biocides that are effective against bacteria and other microorganisms present in biological pathogens. For example, biocides that may be used in one or more embodiments of the invention include tetrakishydroxymethyl phosphonium sulfate (THPS), benzalkonium chloride (BAC) or other quaternary ammonium salts. Preferably, the composition is substantially free of triclosan.

In one or more embodiments, the composition of the present invention comprises a biocide blend—for example a blend of THPS and BAC, a chemical agent binding protein—for example bovine serum albumin, and an enzyme active against organophosphorous compounds—for example OPAA. In one or more embodiments, the composition further comprises a foam forming material, for example a commercial fire fighting foaming material such as Kidde Fire Fighting Foamer. In one or more embodiments, the composition of the invention comprises a basing agent (such as, for example, potassium hydroxide) for maintaining the pH of the composition between about 7.0 and 9.0. In one or more embodiments, the composition may further comprise trace amounts of one or more metals, for example manganese in the form of manganese chloride.

In one or more embodiments, the composition has at least one biocide, and, more typically, a blend of two or more biocides. Biocides function to kill, disable, or neutralize biological pathogens. Known biocides include, for example, benzalkonium chloride (BAC), tetrakishydroxymethyl phosphonium sulfate (THPS), triclosan (2,4,4′-trichloro-2′-hydroxydiphenyl ether), streptomycin, sodium omadine, dichlorophen, and methylene bisthiocyanate. Preferred biocides are BAC, THPS, and a combination thereof. Although limited amounts of triclosan can be employed, a preferred composition will be substantially free of triclosan due to its limited solubility in water and stability problems. The biocide(s) is present in an amount effective to kill, disable, or neutralize the target biological pathogens(s) either alone or in conjuction with other ingredients. Total biocide content preferably ranges from about 0.1 to 20 wt %, more preferably from about 0.5 to about 10 wt %, and most preferably from about 1 to 6 wt % based on the weight of the composition.

Optionally, enzymes may be incorporated in the composition. Enzymes function to assist in killing, disabling, or neutralizing biological and chemical pathogens. Preferred enzymes are those that are active against organophosphorous toxins. Such enzymes may include, for example, organophosphate hydrolase (OPH), organophosphorous acid anhydrase (OPAA), glucose oxidase, lysing enzyme, lysozyme, protease, chitinase, lysostaphin, mutanolysin, collagenase, SynthaCLEC-GO (Altus, Inc.), PeptiCLEC-TR (Altus, Inc.), and combinations thereof Preferred enzymes are OPAA or OPH, with OPAA being particularly preferred.

The enzyme(s) is present in an amount effective to assist the biocide(s) in killing, disabling, or neutralizing the target biological pathogens(s) either alone or in conjunction with other ingredients. Enzymes are typically employed at about 0.1 to about 350 g/L (grams per liter of composition). Preferably, enzymes are employed at from about 0.3 to 12 g/L. “g/L” refers to grams of enzyme per liter of single strength foam solution. The composition is preferably prepared such that it has a suitable pH level for optimal decontamination. Preferably, the composition is prepared such that is has a pH level ranging from about 6 to about 9. Any suitable known basing agent or alkaline buffer may be employed. Useful inorganic basing agents include phosphates and carbonates. Useful inorganic basing agents include potassium monophosphate and potassium hydroxide. A useful organic basing agent is tris(hydroxymethyl)amino methane.

Optionally, a binding protein(s) may be incorporated in the composition. The binding protein functions to bind and preferably denature or otherwise disable chemical agents or pathogens. The binding protein may be selected from those known in the art. For example, suitable proteins may include albumin, e.g., bovine serum albumin (BSA), acetylcholinesterase (AChE), butyl cholinesterase (BuChE), cholinesterase (ChE), chymotrypsin, trypsin, chymotrypsinogen, trypsinogen, urokinase, esterase, carboxylesterase, thrombin, Factor VII_{A}, Factor X_{A}, kallikrein, prekallkrein, Na/K-ATPase, papain and alkaline phosphatase. A preferred protein is bovine serum albumin.

The protein(s) is present in an amount effective to assist in killing, disabling, or neutralizing the target chemical or biological pathogens(s) in conjunction with other ingredients, such as the biocide(s) and enzyme(s). When employed, proteins are added to the present composition at levels of from about 0.001 to about 40 g/L, more preferably at about 0.005 to 10 g/L, and more preferably at about 0.01 to 1.0 g/L. “g/L” refers to grams of protein per liter of composition.

Optionally, trace metals may be added to enhance enzyme activity. Useful trace metals include, for example, MnCl₂, MgCl₂, CaCl₂, CdCl₂, CoCl₂, CuCl₂, FeCl₂, and potassium monophosphate. Particularly preferred trace metals are manganese chloride and potassium monophosphate. Manganese chloride is particularly useful in enhancing the activity of OPAA enzyme. Preferably, trace metal salts are added in amounts from about 0.5 to 2.5 mM (millimoles) in the composition. More preferably, trace metal salts are added in amounts from about 0.5 to 1.5 mM.

In one or more embodiments, the composition is aqueous, allowing it to be sprayed or applied to the site of chemical or biological contamination. The composition typically has about 70 wt % or more (about 70 wt % to less than 100 wt %) water. Certain embodiments have about 85 wt % to about 95 wt % water.

Surfactants function to stabilize and, optionally, emulsify, hydrophobic ingredients in the aqueous phase of the composition. Hydrophobic ingredients can include, for example, biocides, hydrocarbon solvents, and foam stabilizers. Useful surfactants include anionic, nonionic, and amphoteric/zwitterionic surfactants. Surfactants will typically be present at about 20 wt % or less, more typically about 10 wt % or less, and most typically up to about 0.8 to about 5 wt % based on the total weight of the composition.

Anionic surfactants include those surfactants in which the charge on the hydrophobe is negative. Strong anionic surfactants can exhibit high foaming characteristics and can be added as foaming agents. An anionic surfactant(s) is present in an amount sufficient to impart a foam form to a foamable composition. Useful anionic surfactants include, but are not limited to, salts of acylamino acids, salts of carboxylic acids, salts of phosphoric acids, salts of sulfonic acids, and sulfuric acid esters. Examples of useful anionic surfactants are sulfosuccinates, sarcosinates, alpha-olefin sulfonates, sarcosines and fatty alcohol sulfates. Additional anionic surfactants are disclosed in the International Cosmetic Ingredient Dictionary and Handbook, 9^{th} ed., vol. 4, p. 2955-2962, which is incorporated herein by reference. A preferred anionic surfactant is sodium laureth sulfate. Preferred anionic surfactants have an HLB of about 40 to about 53. A preferred anionic cosurfactant is disodium isodecyl sulfosuccinate.

Nonionic surfactants include those that are surface active but carry no charge. Nonionic surfactants may have levels of ethoxylation or propoxylation. Useful non-ionic surfactants include those of the following: alcohols, alkanolamides, amine oxides, esters, and ethers. Examples of useful nonionic surfactants are sorbitan derivatives, fatty alcohol ethoxylates, fatty acid monoisopropanolamides, polyethylene glycol and fatty acid monoethanolamides. Additional nonionic surfactants are disclosed in the International Cosmetic Ingredient Dictionary and Handbook, 9^{th} ed., vol. 4, p. 2955-2962, which is incorporated herein by reference.

Amphoteric surfactants include those having a charge on the hydrophobe that changes as a function of the pH. Amphoteric surfactants carry a positive charge in strongly acidic media and a negative charge in strongly basic media. Amphoteric surfactants carry no charge or are zwitterionic at intermediate pH. Useful amphoteric surfactants include acyl/diallyl ethylenediamines and derivatives and N-Allyamino acids. Additional amphoteric surfactants are disclosed in the International Cosmetic Ingredient Dictionary and Handbook, 9^{th} ed., vol. 4, p. 2955-2962, which is incorporated herein by reference.

In one or more embodiments, a composition system is provided for application to a chemically or biologically contaminated site. The system has a first component, a second component, and optionally a third component. Prior to use, the components are separately provided or packaged together but are kept separated and not allowed to come into contact with each other. The first component is in liquid form while the second and third components are in solid form. Upon use, the second and third components are admixed with the first component. The first component has a biocide or blend of biocides, a foaming agent(s), and water. The first component may optionally have additional ingredients, such as hydrocarbon solvents, foam stabilizers, and trace metals. The second component is a basing agent or alkaline buffer. The optional third component may have ingredients such as a protein, an enzyme, and a neutral buffer.

In one or more embodiments, the composition of the present invention comprises a foamable or foam composition suitable for application to a chemically or biologically contaminated site. The composition includes a biocide of benzalkonium chloride, a basing agent(s), an anionic surfactant(s) (present at about 0.8 wt % or more based on the total weight of the composition), and water. The composition has a pH of about 6 to about 9. In one or more embodiments, the composition includes an additional biocide, for example THPS.

In one or more embodiments, an anionic surfactant(s) is employed as a foaming agent in an amount sufficient to overcome the foam-inhibiting effects of the biocide BAC. BAC has been observed to inhibit foam formation, which is undesirable because a foam form allows effective application of the composition to the site of contamination. The foam form acts as a visual aid to ensure effective coverage of the site of contamination and helps prevent runoff. The amount of anionic surfactant employed will vary depending on the type of anionic surfactant and the amount of BAC present. Based on levels of BAC typically employed, the level of anionic surfactant needed will be about 0.8 wt % or more, preferably about 0.8 to about 9 wt %, and most preferably about 1 to about 2 wt %.

The composition of the present invention is useful in treating a variety of chemical pathogens, such as GA, GB (Sarin), GD, GF, VX, and mustard gas. The composition is useful in treating a variety of biological pathogens, such as Anthrax, the plague, tularemia, cholera, E. coli 0157:H7, and Shigella.

In one or more embodiments, the composition is applied to a site of chemical or biological contamination. The site can take the form of any natural or artificial substrate, surface, or enclosure where contamination is present. For instance, the site may be the ground or turf, street or parking surface, or the inside or outside of a building.

In one or more embodiments, the composition of the invention is prepared in three parts (components) to be mixed just prior to application and as specified in Table 1. Each of the components in its separate state has a long storage life, allowing the components to be stockpiled so as to be ready for use when needed. In one or more embodiments, Part 1 is a pre-mixed foam solution that typically comprises a mixture of a foam forming material and biocides. Part I may also contain various surfactants, emulsifiers, and solvents to enhance solubility of the biocides. Part 2 is a basing powder (for adjusting the pH). Part 3 is an enzyme/protein powder additive that may also contain an additional buffer for maintaining a desired pH.

In Table 1, the “As Supplied” column indicates the approximate percentage by weight of the specified component to be used when mixing each part, while the “Final” column represents the approximate percentage by weight of each component after the three parts have been mixed together. Although specific percentages are given in Table 1, it will be understood by those of skill in the art that the concentrations of each component need not have the exact percentage set forth but can vary in a range around the specified value. Further, although specific components are disclosed, similar components may be substituted or added.

In the embodiment of Table 1, the approximate percentage by weight of Part 1 in the final mixture is 98.465%, the approximate percentage of Part 2 is 0.97%, and the approximate percentage of Part 3 is 0.565%.

In the embodiment of Table 1, the main component of Part 1 is water, which comprises about 93.945% by weight of Part 1. Other ingredients of Part 1 include Tetrakis (hydroxymethyl) Phosphinium Sulfate (“THPS”) (about 3.06% by weight), Sodium Laureth Sulfate (about 1.23%), 2-(2-Butuxyethoxy) Ethanol (about 0.73%), Benzalkonium Chloride (“BAC”) (about 0.5%), Fatty Alcohols, C10-C16 (about 0.21%), Disodium isodecyl sulfosuccinate (about 0.26%), Manganese Chloride (about 0.013%), Potassium Monophosphate (about 0.12%), and Ethanol (about 0.040%). THPS and BAC are biocides. Sodium Laureth Sulfate and the C10-C16 fatty alcohols are surfactants. 2-(2-Butuxyethoxy) Ethanol and Ethanol are a solvents. Disodium isodecyl sulfosuccinate is an emulsifier. Manganese chloride is a trace metal whose purpose is to support the action of the enzymes (of Part 3) when the composition is mixed together. Potassium monophosphate is a fungicide.

Part 2 in the embodiment of Table 1 comprises potassium hydroxide, a basing agent (or buffer) used to adjust the pH of the mixed composition.

Part 3 in the embodiment of Table 1 comprises an enzyme (X-Pro Dipeptidase) and a protein (Bovine Serum Albumin) mixed with a buffer (Tris(hydroxymethyl)amino methane). In the embodiment of Table 1, the buffer makes up about 86.75% by weight of Part 3, the protein about 1.8%, and the enzyme about 11.45%.

It will be understood that the specific percentages set forth in Table 1 are exemplary only and that the percentage of each component can vary in a range around the specified value.

TABLE 1
Composition of One Embodiment

| | |
|---|---|
| As Supplied | Final |

Part 1

| | | |
|---|---|---|
| Water | 93.945% | 92.647% |
| Tetrakis(hydroxymethyl) Phosphinium Sulfate | 3.060% | 2.954% |
| Sodium Laureth Sulfate | 1.230% | 1.188% |
| 2-(2-Butoxyethoxy) Ethanol | 0.730% | 0.705% |
| Benzalkonium Chloride | 0.500% | 0.483% |
| Fatty Alcohols, C10-C16 | 0.210% | 0.203% |
| Disodium isodecyl sulfosuccinate | 0.260% | 0.251% |
| Manganese Chloride | 0.013% | 0.0126% |
| Potassium Monophosphate | 0.012% | 0.0116% |
| Ethanol | 0.040% | 0.039% |

Part 2

| | | |
|---|---|---|
| Potassium Hydroxide | 100% | 0.950% |

Part 3

| | | |
|---|---|---|
| Tris(hydroxymethyl)amino methane | 86.750% | 0.482% |
| X-Pro Dipeptidase (OPAA enzyme) | 11.450% | 0.064% |
| Bovine Serum Albumin | 1.800% | 0.010% |

All percentages are percentages by weight

In one or more embodiments, pre-measured amounts of the three components (i.e. Part 1, Part 2 and Part 3) are provided to a user for mixing on site. In one embodiment, Part 1, which typically is in the form of a liquid, is packaged in pre-measured amounts in a 5-gallon pail, a 50-gallon drum, or a 250-gallon tote. Parts 2 and 3 are separately packaged in sealed plastic containers in pre-measured amounts such that, when combined with the pre-measured amount of Part 1, the resulting mixture will have the desired composition, such as, for example, the composition of Table 1. In one or more embodiments, sufficient room is left in the container that holds Part 1 such that the corresponding containers holding Part 2 and Part 3 can fit inside the Part 1 container for shipping purposes. In one or more embodiments, the Part 1 container is provided with an internal cover or divider that creates a separate space for the Part 2 and Part 3 containers. For example, in an embodiment in which the Part 1 container is a 50-gallon drum, the internal cover/divider may comprise a circular, bowl-shaped vessel, similar in shape to an oil-drain pan, with a lip or flange that forms a seal against the top and/or inside circumference of the 50-gallon drum. The divider is placed in the empty space at the top of the 50-gallon drum, forming a recessed space, separated from the Part 1 component, into which the Part 2 and Part 3 containers may be placed. The 50-gallon drum is sealed with a standard drum lid, resulting in a sealed 50-gallon drum that can be easily shipped, stored and/or transported to a deployment site. In one or more embodiments, an implement for stirring (such as, for example, a wooded or plastic rod) is included in the container as well.

In one or more embodiments, preparation at and application to a decontamination site proceeds as follows. The container containing the three components and the stirring implement (“shipping container”) is transported to the decontamination site. At the site, the shipping container is opened by decontamination personnel, who may be attired in protective clothing, such as biohazard suits. The separate containers containing Parts 2 and 3, respectively, the stirring implement, and any divider that was used to keep the Parts 2 and 3 containers separated from the Part 1 component, are removed.

The stirring implement is assembled if necessary (the stirring implement may comprise two or more parts that need to be assembled for use). The container containing the Part 2 component is opened. The Part 2 component is added to the Part 1 component in the shipping container (which doubles as a mixing vessel) and mixed using the stirring implement. The container containing the Part 3 component is added to the Part 1 and Part 2 mixture, and the resulting mixture is thoroughly mixed using the stirring implement. The composition is now immediately available by use. It can be directly applied to items or surfaces with conventional cleaning applicators (such as sponges or mops). Equipment or tools can be immersed in the composition. The composition can be applied by power washers. The composition can be applied by conventional fire fighting foam application apparatus, such as portable or mobile pumps used with foam making nozzles, compressed air foam systems, and hand carried or wheeled “extinguisher type” canisters and devices.

The example composition of Table 1 is illustrative only and the present invention is not limited to that specific composition. For example, in one or more embodiments, the specific percentages for the individual components can vary by plus or minus 15%, or more, of the values indicated in Table 1. In other embodiments, the specific ingredients specified in Table 1 can be substituted with other ingredients of the same class, provided that such substitution does not adversely affect the stability of the individual components of the composition, or the effectiveness of the composition as a whole.

Table 2 below sets forth a more general embodiment of the present invention than the specific embodiment of Table 1.

TABLE 2
General Embodiment

| Part 1 | Part 2 | Part 3 |
|---|---|---|
| Water | Basing Agent | Biological Media- |
| | | Compatible Buffer |
| Biocide(s) | | Enzyme(s) |
| Foam Former(s) | | Protein(s) |
| Emulsifier(s) | | |
| Surfactant(s) | | |
| Solvent(s) | | |
| Trace Metal(s) | | |
| Fungicide(s) | | |

As shown in Table 2, in the general embodiment of the composition of the invention, the Part 1 component comprises water, one or more biocides, one or more foam forming ingredients, one or more emulsifiers, one or more surfactants (or detergents), one or more solvents, one or more trace metals, and one or more fungicides. Part 2 contains a basing agent. Part 3 contains one or more proteins, one or more enzymes, and a buffer that is compatible with biological media. The basing agent of Part 2 and the buffer of Part 3 cooperate to maintain the pH of the composition resulting from the combination of Parts 1, 2 and 3 at a value of about 8 (typically in the range of 7 to 9). In an alternative embodiment, Parts 2 and 3 can be combined into a single component, provided that the basing agent(s)/buffer(s) used are compatible with the protein(s) and enzyme(s) used.

The emulsifiers and surfactants in the Part 1 component of the composition of the invention enhance the solubility of the biocides in the composition. Solubility is important, because insufficient solubility of the biocides will adversely affect the decontamination effectiveness of the composition as a whole.

Thus, a novel biological decontaminant composition, as well as systems and methods for preparing and applying the composition, have been presented. Although the present invention has been described with respect to particular example embodiments, it will be understood by those of skill in the art that the invention is not limited to those particular embodiments, but includes alternative embodiments that will be evident to those skilled in the art.

1. A composition for application to a chemically or biologically contaminated site, comprising:
benzalkonium chloride;
a basing agent;
an anionic surfactant present at about 0.8 wt % or more based on the total weight of the composition; and
water,
wherein the composition is in foam form, wherein the composition has a pH of about 6 to about 9.

2-12. (canceled)

13. The composition of claim 1, further comprising an enzyme.

14. The composition of claim 13, wherein the enzyme is selected from the group consisting essentially of organophosphate hydrolase, organophosphorous acid anhydrase, glucose oxidase, lysing enzyme, lysozyme, protease, chitinase, lysostaphin, mutanolysin, collagenase, and combinations thereof.

15. The composition of claim 13, further comprising a trace metal and a protein.

16-17. (canceled)

18. A composition system for application to a chemically or biologically contaminated site, comprising:
a first component, the first component having
benzalkonium chloride,
an anionic surfactant present at about 0.8 wt % or more based on the total weight of the composition system, and
water;
a second component, wherein the second component is a basing agent,
wherein the first component and the second component are provided or packaged together but are not in contact with each other.

19-29. (canceled)

30. The composition system of claim 16, further comprising a third component having an enzyme.

31. The composition system of claim 30, wherein the enzyme is selected from the group consisting essentially of organophosphate hydrolase, organophosphorous acid anhydrase, glucose oxidase, lysing enzyme, lysozyme, protease, chitinase, lysostaphin, mutanolysin, collagenase, and combinations thereof.

32. The composition system of claim 30, wherein the first component further has a trace metal.

33-55. (canceled)

56. A composition for applying to biologically contaminated sites, comprising:
water;
at least one biocide;
at least one foam forming ingredient;
at least one basing agent;
at least one solvent;
at least one protein; and
at least one enzyme.

57. The composition of claim 56 further comprising at least one surfactant.

58. The composition of claim 56 further comprising at least one emulsifier.

59. The composition of claim 56 further comprising at least one trace metal.

60. The composition of claim 57 further comprising at least one emulsifier.

61. The composition of claim 60 further comprising at least one trace metal.

62. The composition of claim 56 comprising about 3% THPS.

63. The composition of claim 62 comprising about 1.2% Sodium Laureth Sulfate.

64. The composition of claim 63 comprising about 0.5% BAC.

65. The composition of claim 64 comprising about 0.1% fatty alcohols.

66. A composition for applying to biological contaminated sites, comprising:
a first component comprising water, one or more biocides, one or more foam forming ingredients, and one or more solvents;
a second component comprising a first basing agent; and
a third component comprising a protein, an enzyme, and a second basing agent.

67. The composition of claim 66 wherein said first component further comprises at least one surfactant.

68. The composition of claim 66 wherein said first component further comprises at least one emulsifier.

69. The composition of claim 66 wherein said first component further comprises at least one trace metal.

70. The composition of claim 67 wherein said first component further comprises at least one emulsifier.

71. The composition of claim 70 wherein said first component further comprises at least one trace metal.

72. The composition of claim 66 wherein said first component further comprises about 3% THPS.

73. The composition of claim 72 wherein said first component further comprises about 1.2% Sodium Laureth Sulfate.

74. The composition of claim 73 wherein said first component further comprises about 0.5% BAC.

75. The composition of claim 74 wherein said first component further comprises about 0.1% fatty alcohols.
